(19) United States
(12) Patent Application Publication
(10) Pub. No.: US 2003/0088223 A1
(43) Pub. Date: May 8, 2003

(54) Passive bonds for personal care article

(75) Inventors: Robert Eugene Vogt, Neenah, WI (US); Suzanne Marie Schmoker, Oshkosh, WI (US); Sarah Jane Marie Freiburger, Hortonville, WI (US); Mary Anne Bruemmer-Prestley, Appleton, WI (US)

Correspondence Address: WILHELM LAW SERVICE, S.C., 100 W LAWRENCE ST, THIRD FLOOR, APPLETON, WI, 54911

(73) Assignee: Kimberly-Clark Worldwide, Inc., Neenah, WI (US)

(21) Appl. No.: 09/834,682

(22) Filed: Apr. 13, 2001

(51) Int. Cl. A61F 13/15

(52) U.S. Cl. 604/385.01

(57) ABSTRACT

A personal care article comprising a front portion, first and second lateral sections having inner edges releasably fastened to the front portion and first and second outer portions attached to the front portion, a back portion, and a crotch portion between the front portion and the back portion, Outer edges of the first and second lateral sections are secured to third and fourth side edges of the back portion to form a pant-like article having a waist opening, and first and second leg openings. Strength of the attachment of the outer portions of the lateral sections to the front portion is less than strength of securement of the outer edges of the lateral sections to the back portion, whereby the attachments of the outer portions of the lateral sections to the front portion can be separated from the front portion without separating securement of the first and second lateral sections from the side edges of the back portion.

### BACKGROUND

Various disposable personal care articles exist which are designed to absorb and retain body fluid and/or excrement at the lower portion of the human torso. Examples of such articles include incontinence products such as pants, briefs and other undergarments, baby diapers, feminine care menstrual panties, training pants, etc. While most of these articles perform satisfactorily for their intended purpose, some are difficult to put on and to remove from the body of the wearer. Such articles do not have refastenable structure which allows the article to be easily adjusted during use or to be easily removed from the wearer's body before the product is permanently discarded, while maintaining an underwear-like structure and fit.

Some of today's commercially available products do not conform well to the human body and this poor fit increases the likelihood of waste leakage while the product is being worn.

Some recently developed such personal care article products have refastenable lateral section structures which typically provide size adjustment capability along with corresponding improved fit to the human body, thereby reducing the likelihood of fluid leakage. Inboard portions of such lateral sections can be fastened, released, and refastened along a centrally-disposed area of the front portion of the personal care article thus to facilitate mounting and retaining the personal care article on the wearer. The locus of such fastening and refastening of the inboard portions can typically be selected to accommodate and conform the size of such personal care article to the size of the wearer, within a range of sizes.

Outboard portions of such lateral sections on the front of the personal care article are typically joined with outboard portions of the back portion of the personal care article during article manufacture, thus to form side seams on the personal care article. The front and back portions of the personal care article are thus joined together at such side seams during manufacture of such article, to make the pant-like personal care article.

Conventional manufacturing of such products comprehends securing inboard portions of the lateral sections to the centrally-disposed areas of front portions of personal care article work pieces, and movement of such work pieces along the manufacturing line without securing outboard portions of the lateral sections in the respective work pieces.

The lateral sections are typically made of highly flexible thin fibrous materials. Such movement of the work pieces while such flexible outboard portions of the lateral sections are unsecured to the respective work pieces can be associated with undesired, and uncontrolled flexing or folding of the outboard portions away from the main bodies of the respective work pieces, and thus away from the respective back portions, whereupon the outboard portions of the lateral sections are displaced from, or folded away from, the main bodies of the respective work pieces, and thus cannot be properly assembled to the respective back portions in the work pieces by the machines designed for accomplishing such assembly. As a result, and when such outboard portions are so displaced, the respective work pieces cannot be properly assembled into the desired personal care articles, whereupon both the work pieces and the corresponding lateral sections are typically discarded as waste.

It is an object of the invention to provide a such personal care article wherein outboard edge portions of the lateral sections are at least temporarily secured in the work piece before the outboard edges themselves are permanently attached into the work piece such as to the back portion of what is becoming the resultant personal care article.

It is another object to provide such personal care article wherein outboard edge portions of the lateral sections are secured in the respective work piece before the work piece is moved along the manufacturing line, and after the inboard portions of the lateral sections are secured in the work piece.

It is another object to provide such a personal care article wherein such at least temporary securement is relatively close to, but displaced from the outer edges of the lateral sections, such that the resulting personal care article can be employed on a relatively wider range of body sizes by separating, or not, one or both of the lateral sections from the front portion in selecting the desired size to be associated with use of the personal care article.

### SUMMARY

In general, methods and articles of the invention employ attachment sites at front portions of absorbent articles of the invention to provide a support mechanism whereby the front panel of a respective absorbent article, and/or absorbent article component(s) associated therewith, is prevented from folding, shifting, bunching and/or twisting during one or both the manufacturing process, and putting such absorbent article on a wearer e.g. as a pull-on pant.

In a first family of embodiments, the invention comprehends a personal care article comprising a front portion having a front end, a second end, a first side edge, and a second side edge; first and second lateral sections having inner portions releasably fastened to the front portion and outer portions attached to the front portion; a back portion having a back end, a third side edge, and a fourth side edge; and a crotch portion between the front portion and the back portion. Each of the outer portions of the first and second lateral sections is secured to one of the third and fourth side edges of the back portion to form a pant-like article having a waist opening, and first and second leg openings. Strength of the attachment of the outer portions of the lateral sections to the front portion is less than strength of the securement of the outer portions of the lateral sections to the back portion, whereby the attachments of the outer portions of the lateral sections to the front portion can be separated from the front portion without separating the securement of the first and second lateral sections from the third and fourth side edges of the back portion.

The outer portions of the first and second lateral sections can have respective outer edges extending outwardly of the respective first and second side edges of the front portion whereby the lateral sections are directly secured to respective ones of the third and fourth side edges of the back portion, without intervening material between the lateral sections and said back portion.

An absorbent core can be attached to at least one of the front portion, the back portion, and the crotch portion.

Preferably, the attachment of the outer portions of the lateral sections to the front portion comprises employment of at least one of adhesive bonds, ultrasonic bonds, thermally activated bonds, hook and loop fastening, snaps, buttons, tapes, needle punching, and piercing.

In preferred embodiments, the first and second lateral sections generally overlie at least a major fraction of the front portion.

In some embodiments, each of the first and second lateral sections has an inner edge, and the inner edges are aligned in abutting relationship, each with respect to the other. Such inner edges can be joined together by a line of weakness.

In a second family of embodiments, the invention comprehends a personal care article comprising a front portion having a front end, a second end, a first side edge, and a second side edge; first and second lateral sections, each having an inner portion releasably fastened to the front portion, and an outer portion attached at one or more attachment sites to the front portion; a back portion having a back end, a third side edge and a fourth side edge; and a crotch portion between the front portion and the back portion. Each of the outer portions of the first and second lateral sections is positioned on the front portion for direct securement to one of the third and fourth side edges of the back portion to form a pant-like article which has a waist opening, and first and second leg openings. The strength of attachment of the outer portions of the lateral sections to the front portion is sufficiently weak that the attachments can be separated from the front portion without substantially fracturing the front portion.

In preferred embodiments, the attachment, at the attachment sites, between the outer portions of the lateral sections and the front portion comprises employment of at least one of adhesive bonds, ultrasonic bonds, hook and loop fastening, and thermally activated bonding.

In a third family of embodiments, the invention comprises a method of assembling a pant-like personal care article having a front portion defining a front end, a second end, a first side edge, and a second side edge, a back portion having a back end, a third side edge and a fourth side edge, and a crotch portion between the front portion and the back portion. The method comprises releasably fastening inner portions of first and second lateral sections to the front portion; attaching outer portions of the first and second lateral sections to the front portion adjacent the respective first and second side edges of the front portion; and securing the outer portions of the first and second lateral sections to the back portion, with the strength of securement being greater than the strength of attachment of the outer portions of the lateral sections to the front portion, to form the pant-like article, having a waist opening and first and second leg openings. The attachments of the outer portions of the lateral sections to the front portion are such that the outer portions of the lateral sections can be separated from the front portion without separating the securement of the first and second lateral sections from the third and fourth side edges of the back portion.

In some embodiments, the method includes attaching the outer portions of the first and second lateral sections to the front portion adjacent the respective first and second side edges of the front portion before attaching the outer portions of the first and second lateral sections to the back portion.

In preferred embodiments, the method includes attaching the outer portions of the lateral sections to the front portion by employing at least one of adhesive bonds, ultrasonic bonds, hook and loop fastening, and thermally activated bonding.

In preferred embodiments, the method includes attaching the outer portions of the lateral sections to the front portion with sufficiently weak attachments whereby such attachments can be separated from the front portion without substantially fracturing the front portion.

In a fourth family of embodiments, the invention comprehends a method of using a pant-like personal care article having a waist opening and first and second leg openings. The personal care article has a front portion defining a front end, a second end, a first side edge, and a second side edge, a back portion having a back end, a third side edge, and a fourth side edge, and a crotch portion between the front portion and the back portion. Inner portions of first and second lateral sections are releasably fastened to the front portion, and outer portions of the first and second lateral sections are secured to the back portion. The method comprises attaching outer portions of the first and second lateral sections to the front portion adjacent the respective first and second side edges of the front portion with the strength of attachment being less than the strength of securement of the outer portions of the lateral sections to the back portion; and separating the outer portion of at least one of the first and second lateral sections from the attachments to the front portion adjacent the respective one of the first and second side edges without separating the respective outer portion of the respective lateral section from the respective securement to the back portion.

The method preferably includes attaching the outer portions of the lateral sections to the front portion by employing at least one of adhesive bonds, ultrasonic bonds, hook and loop fastening, and thermally activated bonding.

### BRIEF DESCRIPTION OF THE DRAWINGS

FIG. 1 shows a plan view of a personal care article work piece of the invention having lateral sections wherein outer portions are secured to the front portion inside of the outer edge of each lateral section.

FIG. 2 shows a cross-sectional view of the personal care article taken at line 2-2 of FIG. 1.

FIG. 3 shows a perspective view of an assembled personal care article after the first and second lateral sections are secured to the back portion and the waist and leg portions are opened up to illustrate respective such openings.

FIG. 4 shows a perspective view as in FIG. 3 illustrating the lateral sections as a single element prior to separation, connected to each other by a line of weakness and to the front portion by securements in outer portions thereof.

FIG. 5 shows a cross-sectional view of the personal care article taken at line 5-5 of FIG. 4

The invention is not limited in its application to the details of construction or the arrangement of the components set forth in the following description or illustrated in the drawings. The invention is capable of other embodiments or of being practiced or carried out in other various ways. Also, it is to be understood that the terminology and phraseology employed herein is for purpose of description and illustration and should not be regarded as limiting. Like reference numerals are used to indicate like components.

### DESCRIPTION OF THE ILLUSTRATED EMBODIMENTS

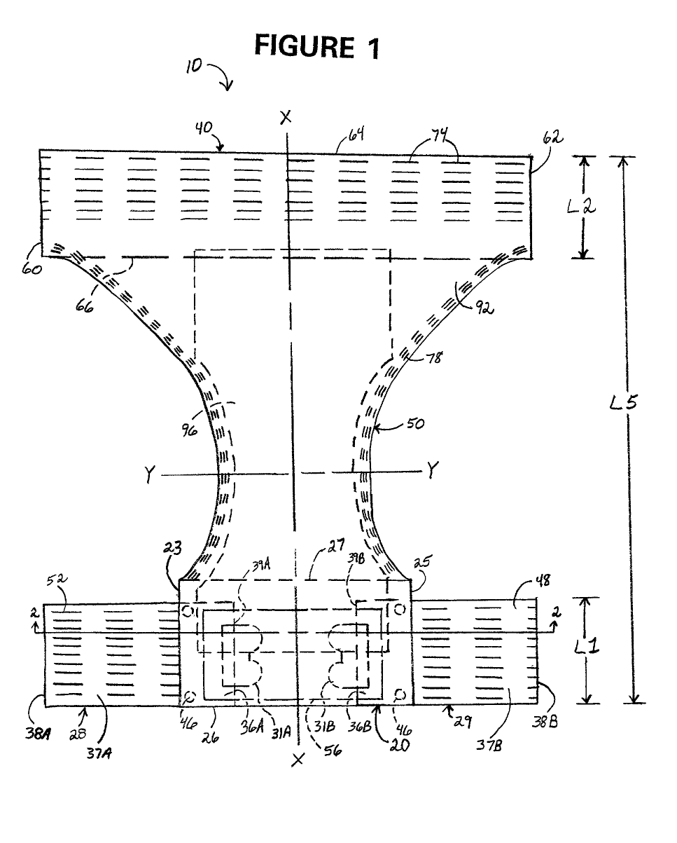

Referring to FIGS. 1, 3, and 4, a personal care article 10, having a refastenable and size-adjustable structure is shown. As shown in FIG. 1, personal care article 10 is preferably a disposable article having a longitudinal axis X-X, hereafter “X-axis”, and a transverse axis Y-Y, hereafter “Y-axis”. The personal care article includes a front portion 20, a back portion 40, and a crotch portion 50 which extends between front portion 20 and back portion 40.

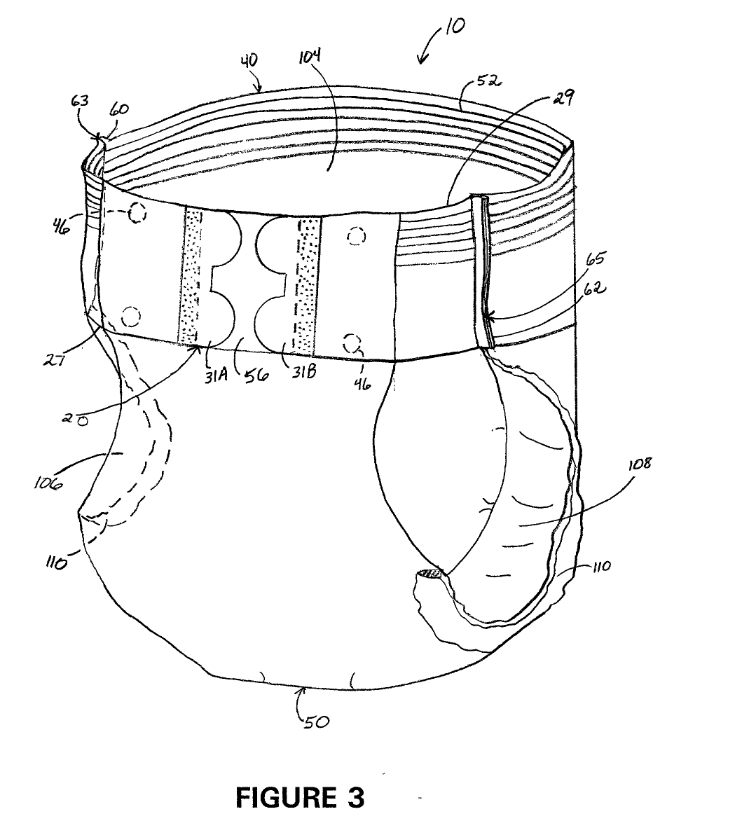

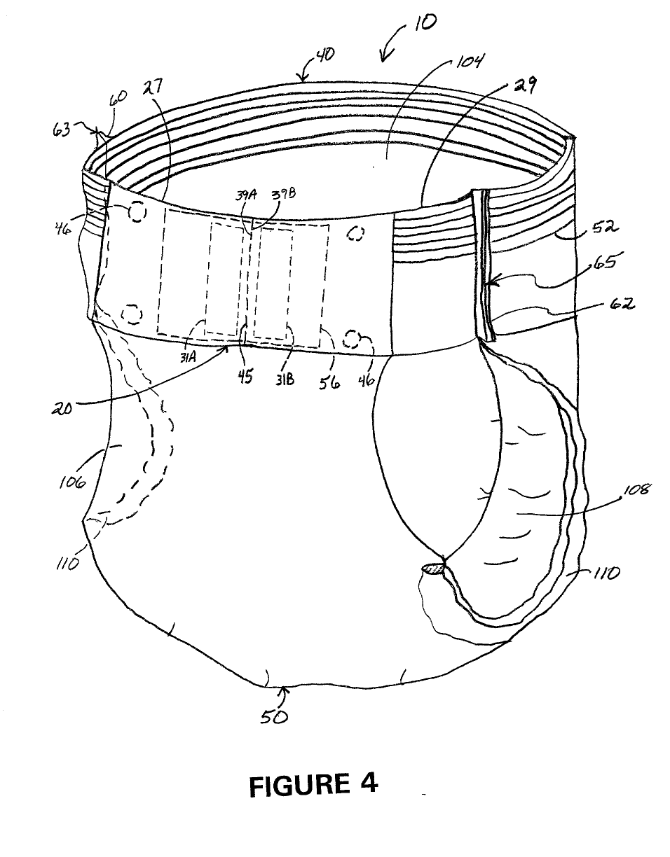

Referring to FIG. 1, front portion 20, which will be aligned around a portion of the front torso of a wearer's body in use, as suggested in FIGS. 3 and 4, includes an front end 26, an second end 27, a first side edge 23, a second side edge 25, and a fastener-receptive area 56. A first lateral section 28 and a second lateral section 29 are connected to front portion 20 via various means described herein.

First lateral section 28 includes an inner portion 36A having an inner edge 39A, and an outer portion 37A having an outer edge 38A. Inner portion 36A of first lateral section 28 comprises a fastener 31A affixed at or near inner edge 39A wherein at least a portion of fastener flap 31A overlaps inner edge 39A to provide a cooperative engagement relationship with fastener-receptive area 56.

Similarly, second lateral section 29 includes an inner portion 36B having an inner edge 39B, and an outer portion 37B having an outer edge 38B. Inner portion 36B of second lateral section 29 also comprises a fastener 31B affixed at or near inner edge 39B wherein at least a portion of fastener flap 31B overlaps inner edge 39B to provide a cooperative engagement relationship with fastener-receptive area 56.

First and second lateral sections 28 and 29 are releasably fastened to fastener-receptive area 56 of front portion 20 via fasteners 31A and 31B. Fasteners 31A and 31B provide refastenable structure which can be repeatedly released, moved laterally to adjust for desired size change, and then refastened at the adjusted size.

As to each lateral section, the respective fasteners are preferably, but not necessarily, aligned, with respect to each other, generally parallel to the X-axis. In some embodiments, fasteners 31A and 31B are incorporated as adjunct elements of the lateral sections, and can be arranged in an abutting relationship to one another (FIG. 4) and second edges 26A of the first and second lateral sections are aligned adjacent outer front portion end 26.

Fasteners 31A and 31B, as illustrated herein, define attachment structures which, e.g. in combination with fastener-receptive area 56, or the like, can be repeatedly fastened, released, adjusted and re-fastened. Acceptable embodiments of fasteners 31A and 31B can include any material capable of forming cooperative engagement relationships with the respective material used for fastener-receptive area 56. For example and without limitation, such acceptable fastener materials are adhesives, cohesives, tapes, mechanical fasteners such as buttons and corresponding buttonholes, snaps and the like, as well as other fasteners which can be repeatedly fastened and released known to those skilled in the art. Mechanical hook and loop fasteners are preferred because of their associated durability and consumer acceptance.

Preferably, fasteners 31A and 31B are structured for easy grasping by a user to pull the respective fastener away from the fastener receptive area of the front portion to thereby release the fastener, and thus the respective lateral section, away from the front portion.

Fastener-receptive area 56 can comprise a patch of landing zone material which can be affixed to front portion 20 by e.g. applying a suitable adhesive to the landing zone material patch or to front portion 20 and affixing the fastener-receptive area to front portion 20 using e.g. a cut-and-place applicator.

Referring to FIGS. 1 and 3, while the patch of landing zone material comprising fastener-receptive area 56 is illustrated as being a one-component, generally rectangular-shaped, piece of landing zone material, the fastening area can be defined by a variety of shapes and sizes, and any desired number of separate components.

Fastener-receptive area 56 can be constructed from a material which preferably has e.g. loop properties or hook material properties. In the alternative, any material which can form a cooperative relationship with desired fastener materials, such as those suggested above, to provide repeatable fastening and releasing properties, is suitable for use as, or in place of, fastener-receptive area 56.

In some embodiments, front portion 20 comprises an outer cover layer made of material which demonstrates landing zone properties capable of forming engagement relationships with aforementioned fastener materials. In such embodiments, no separate patch of fastening material need be applied to front portion 20, since at least a portion, and up to the entirety, of the major surface of front portion 20 already includes fastening area properties. Thus, in such embodiments, fasteners 31A and 31B can form a cooperative refastenable engagement relationship directly with the outer cover layer which serves as the fastener-receptive area of such embodiment.

Therefore, fastener-receptive area 56 may or may not have distinct physical edges, depending on whether the fastening properties desired to be performed thereby (i) are provided by distinct separate e.g. web element(s) or (ii) are integral with a surface of front portion 20.

FIGS. 1, 3, and 4 illustrate attachment of lateral sections 28 and 29 to front portion 20 at attachment sites 46. Two attachment sites 46 are illustrated on outer portions 37A and 37B of respective lateral sections 28 and 29, adjacent respective side edges 23 and 25 of front portion 20. The primary purpose of attachment sites 46 is to control lay of the respective outer portions of the lateral sections against front portion 20 so as to assist in forming side seams as at 63 and 65 between back portion 40 and the respective lateral sections.

Attachment sites 46 can be structured according to any means known to those skilled in the art for affixing layer structures to each other which are consistent with the materials recited herein for the respective elements. Thus, for example, attachment sites 46 can be dots of adhesive placed on either or both of the front portion and the lateral sections. In the alternative, the front portion and/or the respective lateral sections can be softened and bonded together using heat or ultrasonic energy to make the attachment of first and/or second lateral sections 28 and 29 to front portion 20. Hook and loop fastening can also be used to effect the affixation illustrated as attachment sites 46.

The number and size of the attachment sites is of little importance so long as the corresponding affixations control lay of the outer portions of the lateral sections while enabling separation of the lateral sections from the front portion to accommodate size and/or fit adjustments.

Referring to FIG. 1, attachment sites 46 should be located just inwardly of, but adjacent, side edges 23 and 25 of front portion 20, relative to the X-axis. Attachment sites 46 should be designed and constructed so that the attachment sites can be easily broken to enable adjusting the size of personal care article 10 to the size of the wearer, as desired.

Attachment sites 46 should adhere the front portion and the respective lateral sections to each other with sufficient strength to enable the user to use personal care article 10 as an undergarment without accidental and premature breaking of the bonds at attachment sites 46. Attachment sites 46 should not be so weak as to cause unintentional opening or fracture of the attachment sites under normal stresses of installing the personal care article on a wearer. Attachment sites 46 should be sufficiently weak, however, to enable the user to readily break the attachment sites selectively on one or both of lateral sections 28 and 29, thereby to enable a user to expand the waist opening of the personal care article e.g. to adjust the fit of the personal care article without fracturing front portion 20.

Fracturing the front portion is defined as tearing a substantial distance into the front portion, e.g. from the outer cover side, such as to expose the body-side liner and/or the absorbent core, underlaying the outer cover. Such fracturing can cause so much damage to the personal care article that the article may then be no longer suitable for use, and may be disposed of without being employed in its intended use on a wearer's body.

FIGS. 4 and 5 illustrate another embodiment of the invention, wherein personal care article 10 uses temporary attachment of the first and second lateral sections 28 and 29 to front portion 20, as well as to each other. In this embodiment, first and second lateral sections 28 and 29 are joined to each other by a line of weakness 45 when assembled into the personal care article. Line of weakness 45 can comprise, for example, a line or lines of weakness which, when broken, define inner edges 39A and 39B (FIG. 4) which generally correspond to inner edges 39A and 39B of FIGS. 1 and 3. Line of weakness 45 is designed and constructed to be readily broken, but only if and when desired by a user.

Such line of weakness can comprise, for example, a line of breakable perforations or cuts. Lines of weakness are designed and constructed to be readily broken, but only if and when desired by a user, care-giver, or manufacturer.

If desired, line of weakness 45 can be broken before the personal care article is placed around the torso of the wearer, in order to improve body fit, for example by changing sizing of personal care article 10 by changing location of one or both of inner edges 39A and 39B. As in the embodiments of FIGS. 1 and 3, the user can leave inner portions 36A and 36B of the lateral sections affixed to the fastener-receptive area of the front portion while putting the personal care article on the body of a wearer; and can subsequently displace the lateral sections, either singly or in combination e.g. to subsequently adjust the fit on the wearer. In the embodiments of FIG. 4, such displacement correspondingly breaks the bonds at perforation line 45.

There are at least two significant advantages to employing line of weakness 45. First, only one element need be handled in assembling the combination of lateral sections 28 and 29 to a respective workpiece, thus potentially simplifying assembly. Second, since lateral sections 28 and 29 are joined to each other at line of weakness 45, the lateral sections are automatically aligned with each other on the work piece when applied to the work piece as a laid out, unfolded, web element.

As in the previous embodiments, first and second lateral sections 28 and 29 of personal care article 10 are attached to front portion 20 of the personal care article by using releasable bonds sites 46.

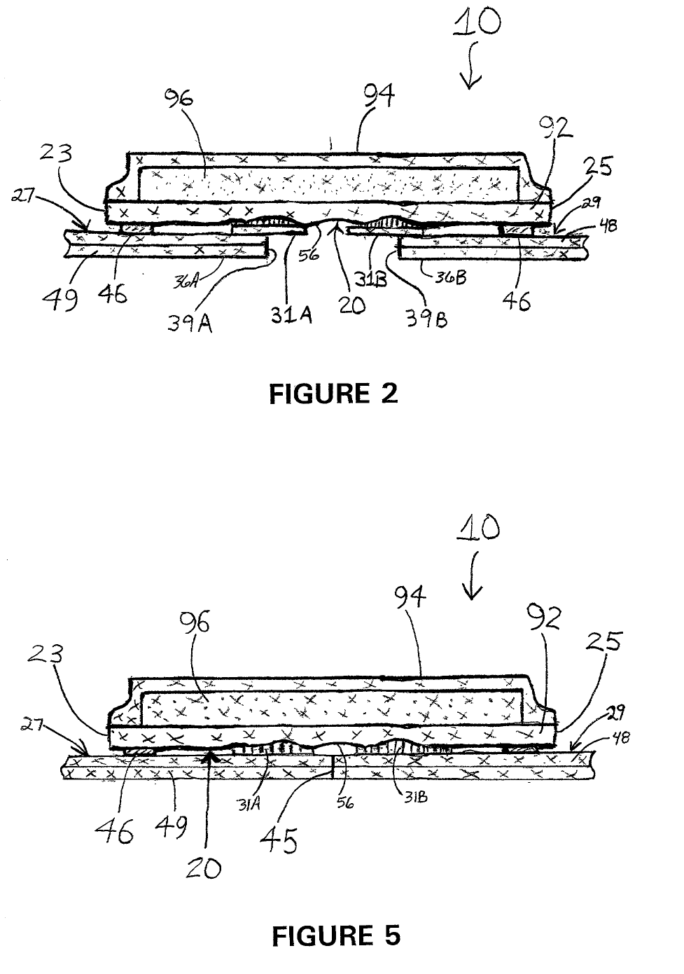

Now referring back to FIG. 2, each of first and second lateral sections 28 and 29 can be formed from a single layer of e.g. web material or can comprise two or more layers generally bonded together so as to co-act as a unitary element. When the first and second lateral sections comprise two or more layers, inner layer 48, which is disposed toward the wearer's body, is preferably soft, and liquid-permeable. Outer layer 49 disposed outwardly of the wearer's body, from layer 48, is preferably liquid-impermeable. In some embodiments first and second lateral sections 28 and 29 are liquid-permeable and are constructed from soft material such as non-wovens.

Elasticity can be imparted to first and second lateral sections 28 and 29 by employing a resiliently extensible material. Such resiliently extensible material can comprise multiple elastics 52, as shown in FIGS. 1 and 3. Such elastic 52 can vary in size, length, and shape. Elongated elastic ribbons, elastic strips, elastic bands, elastic tape having round, flat or other cross-sectional configuration can also be used. Such ribbons, strips, bands, strands, or elastic tape can correspond with a relatively small (e.g. less than 10 percent) fraction of the areas of the lateral sections, up to and including 100 percent of the fraction of the areas of the lateral sections. The greater the fraction of the area covered by the elastic, the greater the opportunity for employing a relatively smaller number of elastic elements. To the extent a contiguous area is to be covered, the elastic can comprise a single element, covering up to 100 percent of the area of the respective lateral sections.

As illustrated in the respective FIGURES, elastic strands 52 provide first and second lateral sections 28 and 29 with extendible properties. Alternatively, first and second lateral sections 28 and 29 can be formed from a material which exhibits or can be modified to exhibit elastic properties. Elastic strands 52 can be formed from e.g. rubber, polyurethane, styrene butylene styrene copolymer, styrene ethylene butylene styrene copolymer, or other elastomeric materials. A typical material is LYCRA® which is commercially available from E. I. Du Pont De Nemours and Company, Willington, Del.

Elastic strands 52 can include for example, two or more strands positioned between inner and outer layers 48 and 49, respectively, and can be held in place by glue, adhesive, cohesive, or other suitable binder. Preferably, elastic strands 52 are arranged in parallel rows relative to one another as illustrated in FIGS. 3 and 4. Other special arrangements of elastic strands 52 can also be used, consistent with desirable fit properties. In some embodiments, the bodyside liner can be between the outer cover and the elastic elements. In yet other embodiments, the outer cover is between the bodyside liner and the elastic elements.

As an alternative to a web or webs containing elastic, one or both first and second lateral sections, can comprise stretch-bonded laminate and/or neck-bonded laminate, both being resiliently stretchable materials void of elastics. Stretch-bonded laminate and neck-bonded laminate are manufactured by Kimberly-Clark Corporation, Neenah, Wis.

Still referring to FIG. 2, lateral sections 28 and 29 are shown overlying (underlying in the orientation illustrated) a substantial portion of front portion 20, e.g. between inner edges 39A and 39B of the respective lateral sections and side edges 23 and 25 of front portion 20. Preferably, first and second lateral sections 28 and 29 overlie the entire outer major surface of front portion 20.

As used herein, the terms “overlying” and “underlying” as applied to lateral sections 28 and 29 are used interchangeably, since the term to be applied depends on the spacial orientation of the personal care article. The important relationship is that front portion 20 is between the respective lateral sections and the body of the wearer.

Additionally, first and second lateral sections 28 and 29 can also be constructed from material which has fastener-receptive properties or can comprise separate pieces of material secured to the lateral sections, wherein the separate pieces of material enable attachment e.g. at attachment sites 46 of the lateral sections onto front portion 20 such as by e.g. hook and loop fastening properties.

In the embodiment illustrated in FIG. 2, inner portions 36A and 36B of first and second lateral sections 28 and 29 are releasably fastened to front portion 20 by fasteners 31A and 31B. In FIGS. 1 and 2, fastener-receptive area 56 on front portion 20, and fasteners 31A and 31B the lateral sections, cooperate to fasten inner portions 36A and 36B of the lateral sections to front portion 20. As previously discussed, front portion 20 can be constructed from a material which has fastener-receptive properties. In the alternative, a separate piece of landing zone material can be secured in the front portion to provide such fastener-receptive properties to the front portion so as to enable cooperative fastening of corresponding first and second lateral sections 28 and 29 to front portion 20 using fastening techniques discussed herein.

The refastenable structure enables the user to release, adjust, and subsequently re-fasten the personal care article onto the body of the wearer. For example, if personal care article 10 is too tight, first and/or second lateral sections 28 and 29 can be released from front portion 20, repositioned for a looser fit, and then refasten at front portion 20 to maintain the looser fit of the personal care article about the body of the wearer.

In FIGS. 3 and 4, elastics have been illustratively omitted from lateral sections 27 and 29 to clarify locations article components, e.g. attachment sites 46. Although elastics are not specifically illustrated in lateral sections 27 and 29 of FIGS. 3 and 4, such lateral sections are to be understood to comprise any herein describe material or composite for constructing lateral sections 27 and 29.

Referring to FIG. 4, first and second lateral sections 28 and 29 are in joined relationship with each other, at the time the personal care article is assembled, by line of weakness 45. In such embodiment, lateral sections 28 and 29 represent a single physical element embodying the generally centrally-located line of weakness 45.

Line of weakness 45 enables placement of both lateral sections 28 and 29 on the front portion, namely assembly of lateral sections 28 and 29 into the work piece, as a single unit during assembly of the personal care article. Such single unit assembly assures proper placement of the lateral sections with respect to each other, and reduces by one the number of elements which must be brought together in assembly of the personal care article. The attachment of lateral sections 28 and 29 to each other at line of weakness 45 is in addition to the releasable fastening of lateral sections 28 and 29 to front portion 20 at fastener-receptive area 56, as well as the attachment of front portion 20 to the lateral sections e.g. at attachment sites 46.

Returning again to FIGS. 1 and 2, first and second lateral sections 28 and 29 have lengths represented by “L1,” of at least about 2 inches (about 5 cm). Length “L1” is measured parallel to the longitudinal central axis X-X. Preferably, for an adult size garment, length “L1” ranges from about 3 inches to about 7 inches (about 7.5 cm to about 18 cm).

Referring now to FIG. 1, back portion 40 has a back end 64, an inner end 66, and third and fourth side edges 60 and 62. Back portion 40 has a length “L2” measured in substantial alignment with the X-axis from back end 64 to inner end 66. Length “L2” is at least about 2 inches (about 5 cm). Preferably, for an adult size garment, length “L2” is from about 3 inches to about 7 inches (about 7.5 cm to about 18 cm). Most preferably, for an adult size garment, length “L2” is from about 6 inches to about 7 inches (about 15 cm to about 18 cm).

Back portion 40 can be formed from an elastic material or an elastic composite, or can contain strands of elastic 74. Elastic 74 can be in the form of elongated elastic strands, ribbons, strips, etc. which extend transversely across the back portion between third side edge 60 and fourth side edge 62. Preferably, at least six strands of elastic 74 are disposed generally parallel to each other, extending generally in directions between edges 60 and 62. The number of such strands can vary from about two to about fifty strands. Preferably, the number of elastic strands ranges from about ten to about forty. Elastic 74 functions to cause back portion 40 of the personal care article 10 to retract about and to conform to the back of the lower portion of the torso of the wearer, and thereby to prevent fluid leakage from occurring at or about the waist opening.

If desired, a single sheet or a number of pieces of resiliently extensible (e.g. elastic) material, can define or extend coextensively with much, most, or all of the projected area defined by back portion 40.

As another exemplary alternative, stretch-bonded laminate and neck-bonded laminate are resiliently stretchable materials which can be used to form back portion 40, typically without further use of elastic strands.

Leg elastics 78 are shown extending generally along peripheral areas of crotch portion 50, following the contour of the personal care article 10, and substantially spanning from front portion 20 to back portion 40. Leg elastics 78 function to gather the material at the side edges of the crotch portion along leg openings 106 and 108. Leg openings 106 and 108 are formed as apertures in the personal care article as outer portions 37A and 37B of first and second lateral sections 28 and 29, respectively, are secured to third and fourth side edges 60 and 62, respectively, of back portion 40 by side seams 63 and 65, thus to form the pant-like article e.g. as illustrated in FIGS. 3 and 4.

Various types of elastic materials are known for use in leg elastics 78. Leg elastics 78 typically provide overall retractive tensions of from about 10 grams to about 400 grams on a given leg opening at stretch-to-stop conditions. Preferably, leg elastics 78 provide tensions of about 50 grams to about 220 grams. More preferably, leg elastics 78 provide tensions of about 80 grams to about 200 grams.

Referring to FIGS. 1 and 2, crotch portion 50 connects the front portion 20, at or near second end 27, to the back portion, at or near inner end 66.

Crotch portion 50 can have an hourglass shape, rectangular shape, square shape, oval shape, or other configuration suitable for receiving and storing body exudates. Crotch portion 50 can also be curved or arcuate as shown in FIG. 1 or can be, irregular, convex or concave in shape. Preferably, crotch portion 50 has an hourglass configuration with the narrowest distance being between side edges occurring at or near the Y-axis. Preferably, crotch portion 50 is symmetric with relationship to the X-axis

Crotch portion 50 can be formed from e.g. a single layer of material, or a combination of two or more layers of material, laminated or otherwise affixed to each other to form a multiple-layer composite. Typically, some of the material of crotch portion 50 is generally a continuation of the materials used in forming the front and/or back portions.

Crotch portion 50 acts as a baffle to prevent body fluid from exiting the personal care article at the crotch portion. At least one layer of the material used to form the crotch portion should be liquid-impermeable and should in any event, and in composite elements if necessary, represent e.g. the entirety of the area of the crotch portion. Polypropylene, polyethylene, or any of the other thermoplastic liquid-impermeable materials works well to provide such liquid-impermeable properties. Preferably, crotch portion 50 is a laminate formed generally from a layer of thermoplastic film or any other material recited for the outer cover, and a layer of e.g. liquid permeable non-woven material or any other material recited for the body side liner.

Referring now to FIGS. 1, 3, and 4, outer portions 37A and 37B (FIG. 1) of first and second lateral sections 28 and 29 are securely attached (FIGS. 3 and 4) to third and fourth side edges 60 and 62, respectively, of back portion 40 to form pant-like article 10. Pant-like article 10 has a waist opening 104 and first and second leg openings 106 and 108. Leg elastics 78 form gathers 110 around leg openings 106 and 108. Personal care article 10 can be sold in either the assembled condition as shown in FIGS. 3 and/or 4, or with fasteners 31A and 31B detached from fastener-receptive area 56 of front portion.

Personal care article 10 typically has an overall length “L5” measured from front end 26 of front portion 20 to back end 64 of back portion 40. Length “L5” is typically about 10 inches (about 25 cm) to about 45 inches (about 140 cm). Preferably, for an adult size garment, length “L5” is about 20 inches (about 51 cm) to about 40 inches (about 102 cm). Most preferably, for an adult garment, length “L5” is about 30 inches (about 76 cm) to about 35 inches (about 89 cm).

Referring to FIG. 1, to create side seams, absorbent article 10 is folded along the Y-axis. The folding of the absorbent article preferably results in outer edge 38A of first lateral section 28 overlying and/or being in close proximity to third side edge 60 of back portion 40. Similarly, the folding of the absorbent article also preferably results in outer edge 38B of second lateral section 29 overlying and/or being in close proximity to fourth side edge 62 of back portion 40. An area of first lateral section 28 at or near outer edge 38A is then secured to, e.g. bonded to, an area of back portion 40 at or near third side edge 60 to develop side seam 63, and an area of second lateral section 29 at or near outer edge 38B is then secured to, e.g. bonded to, an area of back portion 40 at or near fourth side edge 62 to develop side seam 65.

Length “L1” is preferably about equal in size to length “L2”, thereby to provide an aesthetically pleasing refastenable absorbent article 10. However, length “L1” can be sized to be less than, equal to, or greater than length “L2” and still retain the function of side seams 63 and 65.

Referring to FIG. 2, personal care article 10 includes, as a first thin-section element, liquid-impermeable outer cover 92, and as a second thin-section element, liquid-permeable body-side liner 94. A liquid-absorbent core 96 is positioned between outer cover 92 and body side liner 94. Liquid-impermeable outer cover 92, liquid-permeable body-side liner 94, and liquid-absorbent core 96 comprise a base-substrate which serves as a foundation to which other elements of personal care article 10 are directly or indirectly attached.

Various woven and nonwoven web materials can be used for body-side liner 94. For example, body-side liner 94 can be e.g. a meltblown or spunbonded or other non-woven web of polymeric material selected from the group consisting of polyolefins including polyethylenes and polypropylenes, polyesters, and polyamides, and mixtures, copolymers, and blends of such polymeric fibers. Body-side liner 94 can comprise a carded and/or bonded web composed of natural and/or synthetic fibers. The body-side liner can be composed of a substantially hydrophobic material wherein the hydrophobic material is treated with a surfactant or otherwise processed to impart a desired level of wetability and hydrophilicity to such material.

Body-side liner 94 can comprise, for example, nonwoven, spunbonded, polypropylene fabric fabricated from 2.8-3.2 denier fibers, formed into a web having a basis weight of about 22 grams per square meter and a density of about 0.06 grams per cubic centimeter. The fabric is then surface treated with e.g. about 0.3 weight percent of a suitable surfactant. Body-side liner 94 typically comprises a fibrous web defining a multiplicity of small e.g. microporous openings randomly spaced between the fibers and according to location and orientation of the fibers, and extending from a major surface of the web into the interior of the web. Such small openings typically extend through the entirety of the thickness of the web.

Addressing structure, body-side liner 94 can be fabricated using material selected from e.g. the group consisting of porous foams, reticulated foams, apertured polymeric films, polymeric fibers, and natural fibers. Body-side liner 94 can comprise a multiplicity of components or layers which correspond to any of the materials disclosed herein, as well as others known in the art.

It is generally preferred that outer cover 92 of the absorbent article be formed from a material which is substantially impermeable to liquids. A typical outer cover 92 can be manufactured from a thin plastic film or other flexible liquid-impermeable material. For example, outer cover 92 can be formed from a film of polymeric material selected from the group consisting of polyolefins including polyethylenes and polypropylenes, polyesters, and polyamides, and mixtures, copolymers, and blends of such polymeric materials, having thicknesses, for example, of from about 0.012 millimeter to about 0.13 millimeter.

In embodiments where outer cover 92 should have a more cloth-like feel, the outer cover can comprise a polyethylene film having a nonwoven web, such as a spunbonded web of polyolefin fibers, bonded to a surface thereof. For example, a polyethylene film having a thickness of about 0.015 millimeter can have thermally or otherwise bonded thereto a spunbonded web of polyolefin fibers having fiber thicknesses of from about 1.5 to about 2.5 denier per filament, which nonwoven web has a basis weight of e.g. about 24 grams per square meter.

Further, outer cover 92 can be formed of a woven or nonwoven fibrous web which has been constructed and/or treated to impart a desired level of liquid-impermeability to selected regions which are adjacent or proximate absorbent core 96.

Still further, outer cover 92 can optionally be composed of a micro-porous material which permits vapors to escape from absorbent core 96 and through outer cover 92 while preventing liquidous exudates from passing through the outer cover.

One or both of outer cover 92 and body-side liner 94 can comprise a fibrous web defining a multiplicity of randomly-spaced small openings extending from a major surface of the web into the interior of the web. Polymeric material such as the recited polyolefins including polyethylenes and polypropylenes, polyesters, and polyamides, and mixtures, copolymers, and blends of such polymeric materials can be used in either film form or in non-woven fiber form, for one or both of body-side liner 94 and outer cover 92. As to bodyside liner 94, films are apertured films. As to outer cover 92, fibrous webs are impermeable to e.g. aqueous liquid.

Included in the definition of polymeric materials above are all routine, common, normal additives known to those skilled in the art of polymeric materials such as processing aids, chemical stabilizers, compatibilizers e.g. where more than one material is used, fillers, and the like.

Absorbent core 96 suitably comprises hydrophilic fibers, such as a web or matt or loose collection of cellulosic fluff, in combination with a high-absorbency material commonly known as superabsorbent material. Absorbent core 96 preferably comprises a mixture of superabsorbent hydrogel-forming particles and wood pulp fluff. In place of the wood pulp fluff, one can use synthetic, polymeric, meltblown fibers or a combination of meltblown fibers and natural fibers. The superabsorbent material can be substantially homogeneously mixed with the hydrophilic fibers or can be otherwise combined into absorbent core 96.

Alternatively, absorbent core 96 can comprise a laminate of fibrous webs and superabsorbent material or other suitable means of maintaining a superabsorbent material in a localized area. Absorbent core 96 can additionally comprise an uncreped through air dried paper web material known as UCTAD.

Absorbent core 96 can have any of a number of shapes. For example and without limitation, absorbent core 96 can be rectangular, I-shaped or T-shaped. In such products as e.g. diaper-like articles, pants, and the like, absorbent core 96 is preferably narrower in the crotch portion than in the rear portion or the front portion, especially where the crotch portion of the absorbent article is narrower than the rear portion or the front portion of the absorbent article.

The high-absorbency material in absorbent core 96 can be selected from natural, synthetic and modified natural polymers and materials. The high absorbency materials can be inorganic materials, such as silica gels, or organic compounds, such as cross-linked polymers. The high absorbency materials refer to any structure or composition, along with associated process, which renders normally water-soluble materials substantially water insoluble but swellable, whereby absorbent properties are available but the swelled material is substantially immobile after absorbing water-based liquids. Such superabsorbent material can be fabricated by creating e.g. physical entanglement, crystalline domains, covalent bonds, ionic complexes and associations, hydrophilic associations such as hydrogen bonding, and hydrophobic associations, or Van der Waals forces. Two such superabsorbents are DRYTECH® 2035 M and FAVOR® SXM 880. DRYTECH® is available from Dow Chemical Company, Midland, Mich. FAVOR® is available from Stockhausen, Inc., Greensboro, N.C.

Referring to FIG. 3, personal care article 10 can be used in at least two different ways. First, the personal care article, as presented to the customer, can be used as a pull-on pant-like structure, e.g. in the format illustrated in FIG. 3. In such format, waist opening 104 encircles the wearer's torso, and two leg openings 106 and 108 below the waist encircle the wearer's legs. For purposes of this illustration, first and second lateral sections 28 and 29 are, and remain, attached separately to front portion 20, both inwardly as at fasteners 31A and 31B, and outwardly as at attachment sites 46. The pull-on pant-like structure is slipped onto the wearer without releasing fasteners 31A, 31B or attachment sites 46. For example, the wearer's legs are inserted downwardly through waist opening 104 and into and through the respective leg openings 106 and 108, whereby the waist opening encircles the wearer's legs.

The personal care article is then pulled toward the groin region of the wearer such that leg openings 106 and 108 approach the wearer's groin area and waist opening 104 encircle's the wearer's torso, so as to attain a position on the wearer generally associated with wearing of pull-on pant-like articles.

The wearer can adjust the fitting of the pull-on pant-like structure to create a better relative positioning of the waistband about the torso of the wearer, directed toward comfort of the wearer and retention of the personal care article on the wearer, thus to improve the fit.

Further adjusting can be obtained by subsequent grasping, lifting, and pulling back of fasteners 31A and 31B, away from the front portion, and releasing of breakable attachment sites 46, thereby to release lateral sections 28 and 29 from front portion 20. Fasteners 31A and 31B are then refastened to the fastener-receptive area of the front portion, accompanied by desired repositioning of fasteners 31A and 31B so as to achieve a better relationship between adjusted size of the personal care article and size of the wearer. Release and refastening of fasteners 31A and 31B can occur multiple times (e.g. an indeterminate number of times) to enable proper fitting throughout the expected use life of the personal care article. First and second lateral sections 28 and 29 can be adjusted individually or in combination with each other to create a relatively tighter or relatively looser fit.

The second method of using personal care article 10 is to use such article as a diaper-like article. In use as a diaper-like article and before any putting on the prospective wearer, fasteners 31A and 31B are disassociated from the fastener-receptive area of the front portion, and attachment sites 46 are released so as to effectively distance lateral sections 28 and 29 from the front portion, and the front portion is pulled away from back portion 40. The personal care article is then laid on a preferably horizontal surface with the bodyside liner facing upwardly. The lower portion of the torso of the wearer (e.g. infant or adult) is then laid or otherwise moved onto back portion 40 of the personal care article. Front portion 20 is then brought frontwardly between the legs of the wearer and onto the front of the torso of the wearer. Fasteners 31A and 31B of lateral sections 28 and 29 are fastened to the front portion, completing the application of the personal care article onto the wearer. With exception of the disassociation of the fasteners of the lateral sections from the fastener-receptive area of the front portion, those skilled in the art will recognize the instant above description as a known method of installing a diaper-like article on a wearer.

In the embodiments illustrated in FIG. 4, the personal care article, as provided to the customer can be used as a pull-on pant-like structure, as well. As with the article of FIG. 3, such pant-like structure 10 comprises a waist opening 104, which encircles the wearer's torso, and two leg openings 106 and 108 below the waist, which encircle the wearer's legs. The first and second lateral sections 28 and 29 are, and remain, attached to each other, via line of weakness 45, and to the front portion. First and second lateral sections 28 and 29 extend from side seams 63 and 65 to join at line of weakness 45 disposed at the front portion of the personal care article. Line of weakness 45 preferably defines equal widths of lateral sections 28 and 29.

Such pant-like structure is preferably slipped onto the wearer without releasing lateral sections 28 or 29 from front portion 20. For example, as with the example illustrated in FIG. 3, the wearer's legs are inserted downwardly through waist opening 104 and into the respective leg openings 106 and 108, whereby the leg openings encircle the wearer's legs.

The personal care article is then pulled upwardly with respect to the wearer's torso such that leg openings 106 and 108 approach the wearer's groin area and the waist opening 104 encircle's the wearer's torso, so as to attain a position on the wearer generally associated with wearing of pull-on pant-like articles.

The user can adjust the fitting of the pull-on pant-like structure to create a better relative positioning of the waistband about the torso of the wearer, directed toward comfort of the wearer and retention of the personal care article on the wearer, thus to improve the fit. Further modest increase in size can be obtained by pulling apart lateral sections 28 and 29 at line of weakness 45, then grasping, lifting, and pulling fasteners 31A and 31B of lateral sections 28 and 29, respectively, away from the fastener-receptive area of the front portion, and releasing breakable attachment sites 46 without fracturing the front portion and without fracturing side seams 63 and 65. First and second lateral sections 28 and 29 are then refastened to the front portion 20, accompanied by desired repositioning of the lateral sections so as to achieve a better relationship between size of the personal care article and size of the wearer. Release and refastening of first and second lateral sections 28 and 29 can occur multiple times (e.g. an indeterminate number of times) to enable proper fitting throughout the expected use life of the personal care article. The first and second lateral sections 28 and 29 can be adjusted individually or in combination with each other to create a relatively tighter or relatively looser fit.

In the embodiment of FIG. 4, the personal care article can be used as a diaper-like article, as well. In use as diaper-like article, lateral sections are pulled apart at line of weakness 45 and then are pulled away from front portion 20. Attachment sites 46 are thereby broken so as to release the lateral sections to effectively distance lateral sections 28 and 29 from the front portion, and the front portion is pulled away from the back portion. The personal care article is then laid on a preferably horizontal surface with the bodyside liner facing upwardly. The lower portion of the torso of the wearer (e.g. infant or adult) is then laid on back portion 40 of the personal care article. The front portion is brought frontwardly between the legs of the wearer and onto the front side of the torso of the wearer. Lateral sections 28 and 29 are then refastened to front portion 20, completing mounting of the personal care article 10 on the wearer. Except for the removal of the lateral sections from the front portion, those skilled in the art will recognize the instant above description as a known method of installing a diaper-like article on a wearer.

Alternative methods of putting on a diaper-like article of any of the embodiments herein discussed will be obvious to those of ordinary skill in the art. Such alternative methods include, but are not limited to, e.g. putting the diaper-like article on a wearer when such wearer is in a position other than lying down, e.g. standing, and/or when such wearer's slacks/pants are not entirely removed, but rather, lowered to expose the lower trunk and groin region of the wearer.

Those skilled in the art will now see that certain modifications can be made to the inventions and methods herein disclosed with respect to the illustrated embodiments, without departing from the spirit of the instant invention. And while the invention has been described above with respect to the preferred embodiments, it will be understood that the invention is adapted to numerous rearrangements, modifications, and alterations, and all such arrangements, modifications, and alterations are intended to be within the scope of the appended claims.

To the extent the following claims use means plus function language, it is not meant to include there, or in the instant specification, anything not structurally equivalent to what is shown in the embodiments disclosed in the specification.

Having thus described the invention, what is claimed is:

1. A personal care article, comprising:
(a) a front portion having a front end, a second end, a first side edge, and a second side edge;
(b) first lateral section and second lateral section, each of the first and second lateral sections having an inner portion releasably fastened to said front portion, and an outer portion attached to said front portion;
(c) a back portion having a back end, a third side edge, and a fourth side edge; and
(d) a crotch portion between said front portion and said back portion,
each of said outer portions of said first and second lateral sections being secured to one of said third and fourth side edges of said back portion to form a pant-like article having a waist opening, and first and second leg openings,
strength of attachment of said outer portions of said first and second lateral sections to said front portion being less than strength of the securement of said outer portions of said first and second lateral sections to said back portion, whereby attachment of said outer portions of said first and second lateral sections to said front portion can be separated from said front portion without separating the securement of said first and second lateral sections from the third and fourth side edges of said back portion.

2. A personal care article as in claim 1, said outer portions of said first and second lateral sections each having a respective outer edge extending outwardly of the respective first and second side edges of said front portion whereby said first and second lateral sections are directly secured to respective ones of said third and fourth side edges of said back portion, without intervening material between said lateral sections and said back portion.

3. A personal care article as in claim 1, including an absorbent core attached to at least one of said front portion, said back portion, and said crotch portion.

4. A personal care article as in claim 1 wherein the attachment of the outer portions of the lateral sections to the front portion comprises employment of at least one of adhesive bonds, ultrasonic bonds, thermally activated bonds, hook and loop fastening, snaps, buttons, tapes, needle punching, and piercing.

5. A personal care article as in claim 1 wherein said first and second lateral sections generally overlie said front portion.

6. A personal care article as in claim 1 wherein each of said first and second lateral sections has an inner edge, said inner edges being aligned in an abutting relationship, each with respect to the other.

7. A personal care article as in claim 6 wherein the inner edges are joined together by line of weakness.

8. A personal care article, comprising:
(a) a front portion having a front end, a second end, a first side edge, and a second side edge;
(b) first lateral section and second lateral section, each of the first and second lateral sections having an inner portion releasably fastened to said front portion, and an outer portion attached at one or more attachment sites to said front portion;
(c) a back portion having a back end, a third side edge and a fourth side edge; and
(d) a crotch portion between said front portion and said back portion,
each of said outer portions of said first and second lateral sections being positioned on said front portion for direct securement to one of said third and fourth side edges of said back portion to form a pant-like article having a waist opening, and first and second leg openings,
strength of attachment, at said attachment site, of said outer portions of said first and second lateral sections to said front portion being sufficiently weak that such attachments can be separated from said front portion without substantially fracturing said front portion.

9. A personal care article as in claim 8, said outer portions of said first and second lateral sections each having a respective outer edge extending outwardly of the respective first and second side edges of said front portion whereby said first and second lateral sections are directly secured to respective ones of said third and fourth side edges of said back portion, without intervening material between said lateral sections and said back portion.

10. A personal care article as in claim 8, including an absorbent core attached to at least one of said front portion, said back portion, and said crotch portion.

11. A personal care article as in claim 8 wherein the attachment at the attachment sites between the outer portions of the lateral sections and the front portion comprises employment of at least one of adhesive bonds, ultrasonic bonds, hook and loop fastening, and thermally activated bonding.

12. A personal care article as in claim 8 wherein said first and second lateral sections generally overlie said front portion.

13. A personal care article as in claim 8 wherein each of said first and second lateral sections has an inner edge, said inner edges being aligned in an abutting relationship, each with respect to the other.

14. A personal care article as in claim 13 wherein said inner edges are joined together by line of weakness.

15. A method of assembling a pant-like personal care article having a front portion defining a front end, a second end, a first side edge, and a second side edge, a back portion having a back end, a third side edge and a fourth side edge, and a crotch portion between the front portion and the back portion, the method comprising:
(a) releasably fastening inner portions of first and second lateral sections to the front portion;
(b) attaching outer portions of the first and second lateral sections to the front portion adjacent the respective first and second side edges of the front portion; and
(c) securing the outer portions of the first and second lateral sections to the third and fourth side edges of the back portion, with strength of securement greater than strength of attachment of the outer portions of the first and second lateral sections to the front portion, to form the pant-like article, having a waist opening and first and second leg openings,
the attachments of the outer portions of the first and second lateral sections to the front portion being such that the outer portions of the lateral sections can be separated from the front portion without separating the securement of the first and second lateral sections from the third and fourth side edges of the back portion.

16. A method as in claim 15 including attaching the outer portions of the first and second lateral sections to the front portion adjacent the respective first and second side edges of the front portion before attaching the outer portions of the first and second lateral sections to the back portion.

17. A method as in claim 15, including attaching the outer portions of the lateral sections to the front portion by employing at least one of adhesive bonds, ultrasonic bonds, hook and loop fastening, and thermally activated bonding.

18. A method as in claim 15, including attaching the outer portions of the lateral sections to the front portion with sufficiently weak attachments whereby such attachments can be separated from the front portion without substantially fracturing the front portion.

19. A method of using a pant-like personal care article having a waist opening and first and second leg openings, the personal care article having a front portion defining a front end, a second end, a first side edge, and a second side edge, a back portion having a back end, a third side edge, and a fourth side edge, and a crotch portion between the front portion and the back portion, inner portions of first and second lateral sections being releasably fastened to the front portion, outer portions of the first and second lateral sections being secured to the back portion, the method comprising:
(a) attaching the outer portions of the first and second lateral sections to the front portion adjacent the respective first and second side edges of the front portion with strength of attachment less than strength of securement of the outer portions of the lateral sections to the back portion; and
(b) separating the outer portion of at least one of the first and second lateral sections from attachment to the front portion adjacent the respective one of the first and second side edges without separating the respective outer portion of the respective lateral section from the respective securement to the back portion.

20. A method as in claim 19, including attaching the outer portions of the lateral sections to the front portion by employing at least one of adhesive bonds, ultrasonic bonds, hook and loop fastening, and thermally activated bonding.
